(19) United States
(12) Patent Application Publication
(10) Pub. No.: US 2015/0110871 A1
(43) Pub. Date: Apr. 23, 2015

(54) Gastric retentive tablet compositions

(71) Applicant: David Wong, Milpitas, CA (US)

(72) Inventor: David Wong, Milpitas, CA (US)

(73) Assignee: David Wong, Milpitas, CA (US)

(21) Appl. No.: 14/588,925

(22) Filed: Jan. 3, 2015

Related U.S. Application Data
(63) Continuation-in-part of application No. 14/333,735, filed on Jul. 17, 2014.
Application No. 14/333,735 is a continuation-in-part of application No. 14/293,285, filed on Jun. 2, 2014.
Application No. 14/293,285 is a continuation-in-part of application No. 14/324,192, filed on Jul. 6, 2014.

(51) Int. Cl. A61K 31/554; A61K 31/506; A61K 31/351; A61K 31/4709; A61K 38/00; A61K 31/09; A61K 31/7072; A61K 31/4965; A61K 31/485; A61K 31/4439; A61K 9/20; A61K 31/55

(52) U.S. Cl. CPC A61K 31/554; A61K 9/2072; A61K 9/2027; A61K 31/506; A61K 31/351; A61K 31/4709; A61K 31/55; A61K 31/09; A61K 31/7072; A61K 31/4965; A61K 31/485; A61K 31/4439; A61K 38/00
USPC 424/469; 514/275; 514/252.18; 514/460; 514/312; 514/212.07; 514/718; 514/51; 514/255.06; 514/289; 514/338; 514/211.13; 514/1.1

(57) ABSTRACT

The present invention relates to a gastric retentive tablet composition comprising: (1) coated particles essentially consisting of a drug and an amino methacrylate copolymer, (2) a methacrylic acid copolymer and (3) an excipient, wherein items 1, 2, and 3 are blended together, and then compressed into a gastric retentive tablet. Thus, the coated particles (item 1), a methacrylic acid copolymer and the excipient are evenly distributed in the tablet. The excipient is selected from a group consisting of a retarding agent, a binder, a filler, a chelating agent, a diluent, a disintegrant, a lubricant, a colorant, a solubilizing agent, or a mixture thereof. The coated particles (item 1) do not contain methacrylic acid polymer.

### CROSS-REFERENCE TO RELATED APPLICATION

The present application claims benefit of U.S. patent application Ser. No. 14/293,285 filed Jun. 2, 2014; U.S. patent application Ser. No. 14/333,735 filed on Jul. 7, 2014; and U.S. patent application Ser. No. 14/324,192 filed Jul. 6, 2014, which are incorporated herein by reference.

### TECHNICAL FIELD

The present invention relates to a gastric retentive tablet composition comprising: (1) coated particles essentially consisting of a drug and an amino methacrylate copolymer, (2) a methacrylic acid copolymer and (3) an excipient, wherein items 1, 2, and 3 are blended together, and then compressed into a gastric retentive tablet. Thus, the coated particles (item 1), a methacrylic acid copolymer and the excipient are evenly distributed in the tablet. The excipient is selected from a group consisting of a retarding agent, a binder, a filler, a chelating agent, a diluent, a disintegrant, a lubricant, a colorant, a solubilizing agent, or a mixture thereof. The coated particles (item 1) do not contain methacrylic acid polymer.

### BACKGROUND OF THE INVENTION

An extended-release dosage form of a medicine would, in general, improve compliance and therefore an extended-release dosage form has some distinct advantages over the conventional immediate release formulations. In addition, an extended release dosage form would lower the maximum plasma concentration, and this may result in reduced toxic effects. Some drugs are absorbed high in the upper gastrointestinal tract. A gastric retentive tablet is particularly beneficial for delivery of this type of drugs, since the dosage form would be able to keep the drug in the region of absorption for a prolonged period of time.

Monolayer tablets have been commonly used in gastric retentive dosage forms. U.S. Pat. No. 8,668,929 teaches a dosage form comprising an extended release polymer matrix comprising a dose of acetaminophen and a dose of an opioid, wherein the extended release matrix is comprised of a swellable polymer and imbibes fluid after administration to swell to a size sufficient to promote gastric retention of the matrix. U.S. Pat. No. 8,592,481 teaches a gastric retentive dosage form comprising a hydrophilic polymer that upon ingestion swells to a size sufficient to achieve retention of the dosage form in the stomach in a fed mode for a period of at least about five hours.

Member-coated monolayer tablet has also been suggested. U.S. Pat. Nos. 8,580,303 and 8,333,991 teach a dosage form comprises (a) at least one component that contains a gas generating agent and gabapentin, and (b) at least one hydrophilic membrane in the form of a sachet, which contains component (a), and wherein the hydrophilic membrane expands by inflation, floats on the aqueous phase in the stomach, and is permeable to gastric juice. U.S. Pat. No. 8,529,955, U.S. Pat. No. 8,440,232 and U.S. Pat. No. 8,475,813 suggest a dosage form comprising: a core comprising gabapentin and a pharmaceutically acceptable excipient, and a semipermeable membrane surrounding the core, the semipermeable membrane comprising a plasticizer and being permeable to a fluid in an environment of use and substantially impermeable to unsolubilized gabapentin.

Bilayer tablets have been suggested for gastric retentive dosage forms. U.S. Pat. Nos. 8,685,450 8,394,408 and U.S. Pat. No. 8,409,613 describe a drug tablet including a prolonged-release core and an immediate-release layer. While, U.S. Pat. Nos. 7,736,667, 8,329,215 and U.S. Pat. No. 8,043,630 teach a gastric retentive tablet, comprising: (a) a core comprising a first polymeric matrix with said drug dispersed therein, and (b) a shell encasing said core, wherein the shell swells upon imbibition of water to a size large enough to promote retention of the dosage form in a stomach in the fed mode. The shell may contain a drug, but less in amount compared to the core.

There are different types of medications. The limited versions of the gastric retention tablets may not meet the requirements for all medications. It would be beneficial to have other forms of gastric retentive tablets as alternatives.

### BRIEF SUMMARY OF THE INVENTION

The inventor has found a novel gastric retentive tablet composition comprising a drug, an amino methacrylate copolymer, a methacrylic acid copolymer and an excipient; wherein the amino methacrylate copolymer is an acid soluble polymer, and wherein the amino methacrylate copolymer is not soluble in an aqueous medium at pH higher than 5.0.

Accordingly, in one aspect, the present invention relates to a novel gastric retentive tablet comprising coated particles, methacrylic acid copolymer and a excipient, wherein the core of the coated particles essentially consists of a drug, and its coat essentially consists of EUDRAGIT® E. and wherein the excipient is selected from a group consisting of a retarding agent, a binder, a chelating agent, a filler, a diluent, a disintegrant, a lubricant, a colorant, a solubilizing agent, or a mixture thereof. And further, the core of the coated particles does not contain an excipient, and the coat of the coated particles contains only one polymer and the polymer is EUDRAGIT® E.

In a further aspect, the present invention relates to a novel gastric retentive tablet composition, wherein the drug particle is first coated with an amino methacrylate copolymer, and then mixed with methacrylic acid copolymer and other excipients, compressed into a tablet.

### DETAILED DESCRIPTION OF THE INVENTION

### Definitions

“Optional” or “optionally” means that the subsequently described circumstance may or may not occur, so that the description includes instances where the circumstance occurs and instances where it does not.

Singular forms included in the claims such as “a”, “an” and “the” include the plural reference unless expressly stated or the context clearly indicates otherwise. On the other hand, the singular form “ONE” does not include the plural reference.

By “pharmaceutically acceptable” is meant a carrier comprised of a material that is not biologically or otherwise undesirable.

The term “gastric retentive tablet” refers to a tablet which is able to stay in the stomach for 2-4 hours. Tablet dimensions determine if it is a gastric retentive tablet; usually a tablet with a width of 10 mm shows gastric retention. (U.S. Pat. No. 8,377,453) In this invention, the width of the tablet is about 10 mm or longer, thus, it is a gastric retentive tablet and it is also an oral pharmaceutical tablet.

The term, particle, refers to a tiny body of mass. Coated particle is a particle surrounded with a coat. A coated particle has a core (particle) and a coat. A coat is formed by dissolving a polymer in a solvent, and drying the polymer on the particle surface.

The term “core” refers to the central part of a coated particle, its composition is different from the coat of the same coated particle. There are two types of coated particles in the application. The core of Type 1 coated particle essentially consists of a drug; it contains no excipient. The coat of Type 1 coated particle essentially consists of EUDRAGIT® E, it does not contain other polymers. The core of Type 2 coated particle essentially consists of an excipient; it does not contain a drug. The coat of Type 2 particle essentially consists of EUDRAGIT® L.

### The Invention

The present invention provides a gastric retentive tablet composition and methods for preparing such composition. There are four methods for preparing the particles of this embodiment. The first method comprises: (1) suspending drug particles in a liquid to form a drug suspension, (2) dissolving EUDRAGIT® E in a solvent to form EUDRAGIT® E solution, (3) adding the EUDRAGIT®E solution into the drug suspension of Step (1), and (4) drying the mixture of Step (3) into particles. The second method comprises: (1) suspending drug particles in a fluid-bed, (2) dissolving EUDRAGIT® E in a solvent to form EUDRAGIT® E solution, (3) spraying EUDRAGIT® E solution onto the drug particles of Step (1), and (4) drying particles. The third method comprises: (1) suspending drug particles in a EUDRAGIT® E solution, and then (2) spray drying the mixture to form coated particles. The fourth method comprises: (1) mixing drug particles in a mixer, (2) dissolving EUDRAGIT® E in a solvent to form a EUDRAGIT® E solution, (3) spraying the EUDRAGIT®E solution onto the drug particles of Step (1), and then (4) drying the particles. In this invention. Type 2 coated particles can also be produced by these methods. The coated particles are mixed with other excipients and optionally a drug, compressed into a tablet. The tablet is optionally coated for moisture barrier, taste-masking and/or cosmetic purposes. The gastric retentive tablet may have one or more of the following characteristics: (1) the tablet width is 10.0 mm or larger, and (2) the tablet may swell in an aqueous medium.

The tablet can be formed by direct compression, granulation-compression, pellet-compression or equivalent methods. In direct compression, the particles and other excipients are well-mixed and placed in a press die, compressed to form a tablet. In granulation, a binder solution is sprayed onto a mixture of the “particles” and excipients to form granules. The granules are dried and milled to a desired particle size distribution. Then, the granules are blended with other excipients, and placed in the press-die, compressed to form a tablet. Techniques for making tablets are described in Remington's Pharmaceutical Sciences, (Arthur Osol, editor), 1555-1593(1980). Particle-coating using fluid-bed is described in U.S. Pat. No. 8,282,957. Particle-coating using spray-drying method is described in U.S. Pat. No. 8,911,766. Particle-coating using solvent-evaporation technique is described in U.S. Pat. No. 5,223,369. Some other alternative methods can also be used for particle or particulate coating in this invention.

Accordingly, the present invention provides a gastric retentive tablet comprising a drug, EUDRAGIT®E and an excipient. In this embodiment, the tablet is optionally coated for moisture barrier, cosmetic, easy-swallowing and taste-masking purposes. And, the excipient is selected from a group consisting of a retarding agent, a binder, a filler, a diluent, a disintegrant, a lubricant, a colorant, a chelating agent, a solubilizing agent, or a mixture thereof.

In one embodiment, the gastric retentive tablet composition comprises 3 items: (1) coated particles, wherein each coated particle essentially consists of one core and one coat, wherein the core essentially consists of a drug, wherein the core does not contain an excipient, and wherein the coat essentially consists of EUDRAGIT® E. (2) methacrylic acid copolymer, wherein the methacrylic acid copolymer is soluble in an aqueous medium, only at pH 5.5 or above, and (3) an excipient, wherein the preparation of the gastric retentive tablet comprises the following steps: (1) blending of item 1, item 2 and item 3 to form a blend, and (2) compressing the blend of step (1) into a tablet. In this embodiment, there is no any layer between the drug (the core of the particle) and the EUDRAGIT® E coat. Consequently, there are four methods for preparing the coated particles of this embodiment. The first method comprises: (1) suspending drug particles in a liquid to form a drug suspension, (2) dissolving EUDRAGIT® E in a solvent to form EUDRAGIT® E solution, (3) adding the EUDRAGIT®E solution into the drug suspension of Step (1), and (4) drying the mixture of Step (3) to form coated particles. The second method comprises: (1) suspending drug particles in a fluid-bed, (2) dissolving EUDRAGIT® E in a solvent to form EUDRAGIT® E solution, (3) spraying EUDRAGIT®E solution onto the drug particles of Step (1), and (4) drying particles. The third method comprises: (1) suspending drug particles in a EUDRAGIT® E solution, and then (2) spray drying the mixture to form coated particles. The fourth method comprises: (1) mixing drug particles in a mixer, (2) dissolving EUDRAGIT® E in a solvent to form EUDRAGIT® E solution, (3) spraying EUDRAGIT®E solution onto the drug particles of Step (1), and (4) drying the coated particles. Some other methods for producing coated particles can also be used in such preparation. In this embodiment, the gastric retentive tablet composition optionally further comprises a drug outside of the coated particles (item 1). In this embodiment, the coat of the particles of Item 1 contains only one polymer, and it is EUDRAGIT® E.

In another embodiment, the gastric retentive tablet composition comprises 3 items: (1) coated particles, wherein each coated particle essentially consists of one core and one coat, wherein the core essentially consists of a drug and the coat essentially consists of EUDRAGIT® E and optionally a coating agent, wherein the core of the particle does not contain an excipient, and wherein the coating agent is not a polymer, (2) EUDRAGIT® L, and (3) an excipient, wherein the excipient of Item 3 is selected from a group consisting of a retarding agent, a chelating agent, a binder, a filler, a diluent, a lubricant, a colorant, a solubilizing agent, or a mixture thereof. In this embodiment, the coat of the particles of Item 1 contains only one polymer, and it is EUDRAGIT® E. The coating agent of Item 1 is selected from the group consisting of an anti-sticking agent, a surfactant or a mixture thereof. There is no layer between the drug (i.e. the core of the coated particle) and the EUDRAGIT® E film. And further, the gastric retentive tablet composition may comprise a drug outside of the particles of item 1. In one aspect, the gastric retentive tablet composition further comprises Type 2 coated particles, wherein the coat of Type 2 coated particles essentially consists of EUDRAGIT® L, and wherein Type 2 coated particles do not contain a drug. The core of Type 2 coated particles may essentially consists of EUDRAGIT®E, a gas-forming substance, a water-soluble polymer, or a water-insoluble polymer. In one aspect, the core of the coated particle may essentially consist of one drug. In another aspect, the core of the coated particle may essentially consist of two or more drugs.

In all embodiments, the gastric retentive tablet is a monolayer tablet, and optionally coated a film.

As most of the oral drugs are absorbed in the small intestine, the application of the invention applies to a wide variety of drugs. Examples of such drugs include antihistamines (e.g., dimenhydrinate, diphenhydramine, chlorpheniramine and dexchlorpheniramine maleate), analgesics (e.g., aspirin, codeine, morphine, dihydromorphone, oxycodone, etc.), non-steroidal anti-inflammatory agents (e.g., naproxyn, diclofenac, indomethacin, ibuprofen, sulindac), anti-emetics (e.g., metoclopramide), anti-epileptics (e.g., phenytoin, meprobamate and nitrezepam), vasodilators (e.g., nifedipine, papaverine, diltiazem and nicardipine), anti-tussive agents and expectorants (e.g., codeine phosphate), anti-asthmatics (e.g. theophylline), antacids, anti-spasmodics (e.g., atropine, scopolamine), antidiabetics (e.g., insulin), diuretics (e.g., ethacrynic acid, bendrofluazide), anti-hypotensives (e.g., propranolol, clonidine), antihypertensives (e.g, clonidine, methyldopa), bronchodilators (e.g., albuterol), simethicone, glucosamine, chondroitin, methylsulfonylmethane, steroids (e.g., hydrocortisone, triamcinolone, prednisone), antibiotics (e.g., tetracycline), antihemorrhoidals, hypnotics, psycho-tropics, antidiarrheas, mucolytics, sedatives, decongestants, laxatives, vitamins, stimulants (including appetite suppressants such as phenylpropanolamine), as well as salts, hydrates, and solvates of the same. The above list is not meant to be exclusive. In this invention, the preferred drug candidates are those with significant lower gastrointestinal side effects, such as diarrhea and constipation, and those absorbed mainly in the upper gastrointestinal tract. Examples of individual drugs include, but are not limited to afatinib, axitinib, bosutinib, crizotinib, dasatinib, erlotinilo, fostamatinib, gefitinib, ibrutinib, imatinib, lapatinib, lenvatinib, mubritinib, nilotinib, pazopanib, pegaptanib, ponatinib, regorafenib, ruxolitinib, selumetinib, sorafenib, sunitinib, SU6656 (2,3-Dihydro-N,N-dimethyl-2-oxo-3-[(4,5,6,7-tetrahydro-1H-indo1-2-yl)methylene]-1H-indole-5-sulfonamide), trametinib, tofacitinib, vandetanib, vemurafenib, vismodegib. The examples also include the corresponding varieties such as salt forms and complexes, of these molecules. Finally, the drug is crystalline or amorphous, but in some aspects, crystalline form is preferred.

The amount of excipient employed will depend upon how much active agent is to be used. One excipient can perform multi-functionally. Examples of excipients include but not limited to a retarding agent, a binder, a chelating agent, a filler, a diluent, a disintegrant, a lubricant, a solubilizing agent, a colorant, a chelating agent or a mixture thereof.

Enteric polymer is a polymer soluble in an aqueous medium at pH 5.5 or above. Examples of enteric polymer include but not limited to methacrylic acid copolymer, Type A, methacrylic acid copolymer, Type B, hydroxypropyl methylcellulose acetate succinate (also known as hypromellose acetate succinate), cellulose acetate phthalate, hydroxypropyl methyl cellulose phthalate, polyvinyl acetate phthalate, alginic acid, and sodium alginate. The preferred enteric polymer is methacrylic acid copolymer, Type A, NF, marketed under the brand name of EUDRAGIT® L.

EDURAGIT® E is an amino methacrylate copolymer, it is soluble in most acids, while it is not soluble in an aqueous medium at a pH higher than 5.

Retarding material is a material retarding the drug release or slowing down the matrix erosion. Examples of retarding materials include, but are not limited to, hydroxyalkyl celluloses such as hydroxypropyl cellulose, hydroxypropylmethyl cellulose (2208, 2906 and 2910) or hydroxyethyl cellulose; polyvinyl derivatives such as povidone, crospovidone or polyvinyl alcohol; polyethylene oxides; methyl cellulose; gelatin; polysaccharides such as pregelatinized starch, partially pregelatinized starch, pullulan, dextrin, sodium alginate or gum Arabic, polyethylene glycols and some water-insoluble materials. In the invention, some embodiments specify polyethylene oxide. In fact, polyethylene oxide can be replaced with any high molecular weight polymers, preferably, a water soluble and water-swellable polymer.

Binders include, but are not limited to, starches such as potato starch, wheat starch, corn starch; microcrystalline cellulose; celluloses such as hydroxypropyl cellulose, hydroxyethyl cellulose, hydroxypropyl methylcellulose, ethyl cellulose, sodium carboxy methylcellulose; natural gums like acacia, alginic acid, guar gum; liquid glucose, dextrin, povidone, syrup, polyethylene oxide, polyvinyl pyrrolidone, poly-N-vinyl amide, polyethylene glycol, gelatin, poly propylene glycol, tragacanth, combinations thereof and other materials known to one of ordinary skill in the art and mixtures thereof.

Fillers or diluents, which include, but are not limited to sugar, dextrates, dextrin, dextrose, fructose, lactitol, mannitol, sucrose, starch, lactose, xylitol, sorbitol, talc, microcrystalline cellulose, calcium carbonate, calcium phosphate dibasic or tribasic, calcium sulphate, and the like can be used.

Lubricants may be selected from, but are not limited to, those conventionally known in the art such as magnesium, aluminum or calcium or zinc stearate, polyethylene glycol, glycerol monostearate, glyceryl monosterate, glyceryl behenate, mineral oil, sodium stearyl fumarate, stearic acid, hydrogenated vegetable oils and talc.

Glidants include, but are not limited to, silicon dioxide; magnesium trisilicate, powdered cellulose, starch, talc and tribasic calcium phosphate, calcium silicate, magnesium silicate, colloidal silicon dioxide, silicon hydrogel and other materials known to one of ordinary skill in the art.

The solubilizing agents include, but are not limited to, a surfactant, such as, for example, polysorbate 80 (marketed under the brand name of TWEEN® 80) and the like, a complexing agent, such as, for example, beta-cyclodextrins and the like, a polymer, such as, for example, poloxamer 188, and the like, a co-solvent, such as, for example, methanol and the like. The solubilizing agent may also be an acid o an alkaline, if the solubility of the drug is pH dependent.

Colorants include, but are not limited to, pharmaceutical grade dyes and pigments, red ferric oxide, yellow ferric oxide, titanium dioxide, carbon black, and indigo.

Disintegrants include, but are not limited to, crospovidone, croscarmellose-sodium, sodium starch glycolate, low-substituted hydroxypropylcellulose and other materials known to one of ordinary skill in the art.

Chelating agents include, but are not limited to, alcohol, sodium benzoate, butylated hydroxytoluene, butylated hydroxyanisole and ethylenediaminetetraacetic acid.

Gas-forming agent may be used in the Type 2 coated particles. Examples are metal carbonates or equivalent.

The finished pharmaceutical dosage form of the invention can optionally have one or more coatings such as moisture-barrier film coating, sugar coating and other coatings known in the art. Coating is not considered as a matrix in this invention.

These coating layers comprises one or more excipients selected from the group comprising coating agents, plasticizers, channeling agents, opacifiers, taste-masking agents, fillers, polishing agents, coloring agents, anti-tacking (anti-sticking) agents and the like.

Coating agents (for the finished dosage form) which are useful in the coating process, include, but not limited to, polysaccharides such as maltodextrin, alkyl celluloses such as methyl or ethyl cellulose, cellulose acetate, hydroxyalkylcelluloses (e.g. hydroxypropylcellulose or hydroxypropylmethylcelluloses); polyvinylpyrrolidone, acacia, corn, sucrose, gelatin, shellac, cellulose acetate pthalate, lipids, synthetic resins, acrylic polymers, OPADRY® coating systems, polyvinyl alcohol (PVA), copolymers of vinylpyrrolidone and vinyl acetate (e.g. marketed under the brand name of PLASDONE®) and polymers based on methacrylic acid such as those marketed under the brand name of EUDRAGIT®. These may be applied from aqueous or non-aqueous systems or combinations of aqueous and non-aqueous systems as appropriate.

Additives can be included along with the film formers to obtain satisfactory films. These additives can include plasticizers such as dibutyl phthalate, triethyl citrate, polyethylene glycol (PEG) and the like, channeling agents such as surfactants, short-chain water-soluble polymers, salts and the like, anti-tacking (anti-sticking) agents such as talc, steanc acid, magnesium stearate and colloidal silicon dioxide and the like, fillers such as talc, precipitated calcium carbonate, polishing agents such as Beeswax, carnauba wax, synthetic chlorinated wax and opacifying agents such as titanium dioxide and the like. All these excipients can be used at levels well known to the persons skilled in the art.

### EXAMPLES OF INVENTION

The foregoing examples are illustrative embodiments of the invention and are merely exemplary. A person skilled in the art may make variations and modifications without deviating from the spirit and scope of the invention. All such modifications and variations are intended to be included within the scope of the invention.

### Example 1

Pazopanib hydrochloride particles are mixed with an amino methacrylate copolymer solution, then spray-dried to form coated pazopanib particles. The coated particles, 600 mg, are mixed with methacrylic acid copolymer, 90 mg, polyethylene oxide, 100 mg, microcrystalline cellulose 400 mg and glycerol monostearate 20 mg, and then compressed into a tablet.

Example 2

Imatinib mesylate particles are suspended in the chamber of a fluid-bed. EUDRAGIT® E solution is sprayed onto the particles to form coated imatinib mesylate particles, and dried. The coated particles, 500 mg, and then mixed with another portion of imatinib mesylate, 200 mg, methacrylic acid copolymer, 90 mg, polyethylene oxide, 50 mg, microcrystalline cellulose 400 mg and glycerol monostearate 20 mg; compressed into a tablet.

### Example 3

Lovastatin particles are suspended in a EUDRAGIT® E solution, and then spray-dried. The resulting material, 80 mg, is mixed with methacrylic acid copolymer, 40 mg, microcrystalline cellulose, 800 mg, hydroxypropyl methylcellulose, high viscosity grade, 100 mg, glipizide, 10 mg, and glycerol monostearate, 20 mg, and then compressed into a tablet.

Example 4

Cilostazol particles are suspended in an amino methacrylate copolymer solution, and then spray-dried. Coated cilostazol particles, 80 mg, are mixed with methacrylic acid copolymer, 40 mg, microcrystalline cellulose, 800 mg, hydroxypropyl methylcellulose, high viscosity grade, 100 mg, and glycerol monostearate, 20 mg, and then compressed into a tablet.

### Example 5

Benazepril hydrochloride particles are suspended in a fluid bed chamber, a EUDRAGIT® E solution is sprayed onto the particles. Then the coated particles are dried. The coated benazepril hydrochloride particles are mixed with methacrylic acid copolymer, 40 mg, microcrystalline cellulose, 800 mg, hydroxypropyl methylcellulose, high viscosity grade, 100 mg, and glycerol monostearate, 20 mg, and then compressed into a tablet, wherein the width of the tablet is 10 mm.

### Example 6

Guaifenesin particles are mixed in a chamber. EUDRAGIT® E solution is sprayed. The resulting coated particles are dried in oven. The coated guaifenesin particles are mixed with another. portion of guaifenesin, 800 mg, methacrylic acid copolymer, 10 mg, microcrystalline cellulose, 100 mg, polyethylene oxide, 50 mg, and glycerol monostearate, 20 mg, then compressed into a tablet. The width of the tablet is about 10 mm.

### Example 7

Sofosbuvir particles are mixed in an amino methyacrylate copolymer solution, and then spray-dried. The coated sofosbuvir particles are mixed with methacrylic acid copolymer, 40 mg, microcrystalline cellulose, 800 mg, hydroxypropyl methylcellulose, high viscosity grade, 100 mg, and glycerol monostearate, 20 mg, and then compressed into a tablet.

### Example 8

Glipizide particles are suspended in a fluid bed, sprayed with an amino methyacrylate copolymer solution. The coated glipizide particles are dried in the fluid bed, and then mixed with methacrylic acid copolymer, 40 mg, microcrystalline cellulose, 800 mg, hydroxypropyl methylcellulose, high viscosity grade, 100 mg, and glycerol monostearate, 20 mg, and then compressed into a tablet.

### Example 9

Codeine particles are mixed in vertical mixer. An amino methyacrylate copolymer solution is sprayed very slowly on the codeine particles. The coated particles are then mixed with methacrylic acid copolymer, 40 mg, microcrystalline cellulose, 800 mg, hydroxypropyl methylcellulose, high viscosity grade, 100 mg, and glycerol monostearate, 20 mg, and compressed into a tablet.

### Example 10

Morphine sulfate particles are suspended in the product chamber of a fluid bed. An amino methyacrylate copolymer solution is sprayed onto the particles to form coated morphine sulfate particles. The coated morphine sulfate particles are dried, and then mixed with methacrylic acid copolymer, 40 mg, microcrystalline cellulose, 800 mg, hydroxypropyl methylcellulose, high viscosity grade, 100 mg, and glycerol monostearate, 20 mg, and then compressed into a tablet.

### Example 11

Axitinib particles are suspended in the product chamber of a fluid bed. An amino methyacrylate copolymer solution is sprayed onto the particles to form coated axitinib particles. The coated particles are then mixed with another portion of axitinib particles, methacrylic acid copolymer, 40 mg, microcrystalline cellulose, 800 mg, hydroxypropyl methylcellulose, high viscosity grade, 100 mg, and glycerol monostearate, 20 mg, and then compressed into a tablet.

### Example 12

Crizotinib particles are suspended in the product chamber of a fluid bed. An amino methyacrylate copolymer solution is sprayed onto the particles to form coated criotnib particles. The coated particles are then mixed with methacrylic acid copolymer, 40 mg, microcrystalline cellulose, 800 mg, hydroxypropyl methylcellulose, high viscosity grade, 100 mg, and glycerol monostearate, 20 mg, and then compressed into a tablet.

### Example 13

Dasatinib particles are suspended in the product chamber of a fluid bed. An amino methyacrylate copolymer solution is sprayed onto the particles to form coated dasatinib particles. The coated particles are then mixed with methacrylic acid copolymer, 40 mg, microcrystalline cellulose, 800 mg, hydroxypropyl methylcellulose, high viscosity grade, 100 mg, and glycerol monostearate, 20 mg, and then compressed into a tablet.

### Example 14

An anti-cancer drug, 50 mg is suspended in an amino methyacrylate copolymer solution, and then spray-dried. The coated anti-cancer drug particles are then mixed with methacrylic acid copolymer, 40 mg, microcrystalline cellulose, 800 mg, hydroxypropyl methylcellulose, high viscosity grade, 100 mg, and glycerol monostearate, 20 mg, and then compressed into a tablet.

### Example 15

Codeine particles are suspended in a fluid bed, coated with a solution of amino methyacrylate copolymer and an excipient, and then dried into coated particles. The coated codeine particles are mixed with methacrylic acid copolymer, 40 mg, microcrystalline cellulose, 800 mg, hydroxypropyl methylcellulose, high viscosity grade, 100 mg, and glycerol monostearate, 20 mg, and then compressed into a tablet.

### Example 16

Metformin particles are placed in a mixer, mixed at a high speed. EUDRAGIT® E solution is sprayed onto the metformin powder, dried at 45 deg. C., till moisture content is less than 1.5%. Microcrystalline cellulose particles are suspended in the chamber of a fluid-bed dryer, a diluted EUDRAGIT® L 30 D is sprayed onto the microcrystalline cellulose particles. The coated microcrystalline cellulose particles are dried at 45 deg. C. till its moisture content is less than 1.5%. The coated metformin particles, coated microcrystalline cellulose particles, polyethylene oxide, and magnesium stearate are blended and compressed into a tablet, with a width of 10 mm.

### Example 17

Glipizide particles are placed in a mixer, mixed at a high speed. EUDRAGIT® E solution is sprayed onto the metformin powder, dried at 45 deg. C., till moisture content is less than 1.5%. Microcrystalline cellulose particles are suspended in the chamber of a fluid-bed dryer, a diluted EUDRAGIT® L 30 D is sprayed onto the microcrystalline cellulose particles. The coated microcrystalline cellulose particles and dried at 45 deg. C. till its moisture content is less than 1.5%. The coated metformin particles, coated microcrystalline cellulose particles, polyethylene oxide, an extra portion of metformin and magnesium stearate are blended together for 10 minutes, and compressed into a tablet, with a width of 10 mm.

### Example 18

Afatinib particles suspended in an aqueous EUDRAGIT® E solution are spray-dried to form coated afatinib particles, dried at 45 deg. C. till moisture content is less than 1.5%. EIDRAGIT® E particles are suspended in the chamber of a fluid-bed dryer, a diluted EUDRAGIT® L 30 D is sprayed onto the EUDRAGIT® E particles. The coated EUDRAGIT® E particles are dried at 45 deg. C. till its moisture content is less than 1.5%. The coated afatinib particles, coated EUDRAGIT® E particles, polyethylene oxide, ponatinib and magnesium stearate are blended together for 10 minutes, and compressed into a tablet, with a width of 10 mm.

### Example 19

Sorafenib particles are suspended and mixed in an aqueous solution at pH 7, EUDRAGIT® E is dissolved in a solvent, and added into the sorafenilo suspension. The suspension is stirred overnight to remove the solvent. The whole system is then spray-dried to form coated sorafenib particles. Hydroxypropyl methylcellose (direct compression grade) particles are suspended in the chamber of a fluid-bed dryer, EUDRAGIT® L 30 D is dissolved in a solvent, and sprayed onto the hydroxypropyl methylcellose particles at a slow rate. The coated hydroxypropyl methylcellose particles are dried at 45 deg. C. till its moisture content is less than 1.5%. The coated sorafenib particles, coated hydroxypropyl methylcellose particles, polyethylene oxide, sorafenib and magnesium stearate are blended together for 10 minutes, and compressed into a tablet, with a width of 10 mm.

### Example 20

Omeprazole is suspended in an aqueous EUDRAGIT® E solution, then is spray-dried to form coated oxycodone particles. EUDRAGIT® E particles are suspended in the chamber of a fluid-bed, a diluted EUDRAGIT® L 30 D is sprayed onto the EUDRAGIT® E particles. The coated EUDRAGIT® E particles are dried at 45 deg. C. till their moisture content are less than 1.5%. The coated omeprazole particles, coated EUDRAGIT® E particles, polyethylene oxide, accetaminophen and magnesium stearate are blended together for 10 minutes, and compressed into a tablet, with a width of 10 mm.

### Example 21

Quetiapine fumarate particles are suspended in a chamber of a fluid bed. An EUDRAGIT® E solution is sprayed onto quetiapine fumarate particles to form coated particles consisting of a core and a coat. Microcrystalline cellulose particles are suspended in a chamber of a fluid-bed, a diluted EUDRAGIT® L 30 D is sprayed onto the microcrystalline cellulose particles. The coated microcrystalline cellulose particles are dried at 45 deg. C. till its moisture content is less than 1.5%. The coated quetiapine funnarate particles, coated microcrystalline cellulose particles, polyethylene oxide, another portion of quetiapine fumarate and magnesium stearate are blended together for 10 minutes, and compressed into a tablet, with a width of 10 mm.

### Example 22

Oxycodone particles are suspended in a chamber of a fluid bed. An EUDRAGIT® E solution (optionally with a non-polymeric excipient, e.g. talc) is sprayed onto oxycodone particles to form coated particles consisting of a core and a coat. Microcrystalline cellulose particles are suspended in a chamber of a fluid-bed, a diluted EUDRAGIT® L 30 D is sprayed onto the microcrystalline cellulose particles to form coated microcrystalline cellulose particles. The coated microcrystalline cellulose particles are dried at 45 deg. C. till its moisture content is less than 1.5%. The coated oxycodone particles, coated microcrystalline cellulose particles, polyethylene oxide, oxycodone and magnesium stearate are blended together for 10 minutes, and compressed into a tablet, with a width of 10 mm.

### Example 23

Peptide particles are suspended in a chamber of a fluid bed. An EUDRAGIT® E solution (optionally with a non-polymeric excipient, e.g. talc) is sprayed onto the peptide particles to form coated particles consisting of a core and a coat. Microcrystalline cellulose particles are suspended in a chamber of a fluid-bed, a diluted EUDRAGIT® L 30 D is sprayed onto the microcrystalline cellulose particles to form coated microcrystalline cellulose particles. The coated microcrystalline cellulose particles are dried at 45 deg. C. till its moisture content is less than 1.5%. The coated peptide particles, coated microcrystalline cellulose particles, polyethylene oxide, another portion of the peptide particles and magnesium stearate are blended together for 10 minutes, and compressed into a tablet, with a width of 10 mm.

### Example 24

Particles containing oxycodone and codeine are suspended in a chamber of a fluid bed. An EUDRAGIT® E solution (optionally with a non-polymeric excipient, e.g. talc) is sprayed onto particles to form coated particles consisting of a core and a coat. Microcrystalline cellulose particles are suspended in a chamber of a fluid-bed, a diluted EUDRAGIT® L 30 D is sprayed onto the microcrystalline cellulose particles to form coated microcrystalline cellulose particles. The coated microcrystalline cellulose particles are dried at 45 deg. C. till its moisture content is less than 1.5%. The coated particles, coated microcrystalline cellulose particles, polyethylene oxide, oxycodone and magnesium stearate are blended together for 10 minutes, and compressed into a tablet, with a width of 10 mm.

### Example 25

Sorafenib and lovastatin are co-dissolved in a solvent. The mixture is then dispersed in an aqueous medium to form a suspension. An EUDRAGIT® E solution (optionally with a non-polymeric excipient, e.g. talc) is added to the suspension, mixed well to let solvent evaporate. After the particles are coated, then whole mixture is spray-dried to form coated drug particles. Microcrystalline cellulose particles are suspended in a chamber of a fluid-bed, a diluted EUDRAGIT® L 30 D is sprayed onto the microcrystalline cellulose particles to form coated microcrystalline cellulose particles. The coated microcrystalline cellulose particles are dried at 45 deg. C. till its moisture content is less than 1.5%. The coated drug particles, coated microcrystalline cellulose particles, polyethylene oxide, another portion of sorfenib and lovastatin, and magnesium stearate are blended together for 10 minutes, and compressed into a tablet, with a width of 10 mm.

I claim:

1. A gastric retentive tablet composition comprising 3 items: (1) coated particles, wherein each particle essentially consists of one core and one coat, wherein the core essentially consists of dasatinib, wherein the core does not contain an excipient, wherein the coat essentially consists of EUDRAGIT® E, and wherein there is no layer between the core and the coat, (2) methacrylic acid copolymer, wherein the EUDRAGIT® L is soluble in an aqueous medium, only at pH 5.5 or above, and (3) an excipient, wherein the preparation of the gastric retentive tablet comprises the following steps: (1) blending of item 1, item 2 and item 3 to form a blend, and (2) compressing the blend of step (1) into a gastric retentive tablet composition.

2-5. (canceled)

6. The gastric retentive tablet composition according to claim 1 optionally further comprising dasatinib outside of the coated particles of item 1.

7. The gastric retentive tablet composition according to claim 1, wherein the coated particles of Item 1 contain only one polymer, wherein the only one polymer is EUDRAGIT® E.

8. A gastric retentive tablet composition comprising 3 items: (1) coated particles, wherein each particle essentially consists of one core and one coat, wherein the core essentially consists of dasatinib and the coat essentially consists of EUDRAGIT® E and optionally a coating agent, wherein dasatinib is crystalline, wherein the core of the particle does not contain an excipient, wherein the coating agent is not a polymer, and wherein there is no layer between the core and the coat, (2) EUDRAGIT® L, and (3) an excipient, wherein the coating agent of Item 1 is selected from the group consisting of an anti-sticking agent, a surfactant or a mixture thereof, and wherein the excipient of Item 3 is selected from a group consisting of a retarding agent, a chelating agent, a binder, a filler, a diluent, a lubricant, a colorant, a solubilizing agent, or a mixture thereof.

9-10. (canceled)

11. The gastric retentive tablet composition according to claim 8 further comprising Type 2 coated particles, wherein Type 2 coated particles essentially consists of a core and a coat, wherein the coat essentially consists of EUDRAGIT® L, wherein the core of the Type 2 coated particles essentially consists of EUDRAGIT®E. and wherein Type 2 coated particles do not contain a drug.

12-16. (canceled)

17. A gastric retentive tablet composition comprising 3 items: (1) coated particles, wherein each particle essentially consists of one core and one coat, wherein the core essentially consists of two drugs and the coat essentially consists of EUDRAGIT® E and optionally a coating agent, wherein the two drugs are dasatinib and imatinib mesylate, wherein the core of the particle does not contain an excipient, wherein the coating agent is not a polymer, and wherein there is no layer between the core and the coat, (2) EUDRAGIT® L, and (3) an excipient, wherein the excipient of Item 3 is selected from a group consisting of a retarding agent, a chelating agent, a binder, a filler, a diluent, a lubricant, a colorant, a solubilizing agent, or a mixture thereof.

18. The gastric retentive tablet composition according to claim 17 further comprising Type 2 coated particles, wherein Type 2 coated particles essentially consists of a core and a coat, wherein the coat essentially consists of EUDRAGIT® L, wherein the core of the Type 2 coated particles essentially consists of EUDRAGIT® E, and wherein Type 2 coated particles do not contain a drug.

19. (canceled)
